(19) United States
(12) Patent Application Publication
(10) Pub. No.: US 2023/0035578 A1
(43) Pub. Date: Feb. 2, 2023

(54) CONTROLLED RELEASE OF COMPOUNDS

(71) Applicant: TECHNISCHE UNIVERSITAET MUENCHEN, Munich (DE)

(72) Inventors: Oliver LIELEG, Garching (DE); Ceren KIMNA, Garching (DE); Benjamin WINKELJANN, Landsberg am Lech (DE)

(73) Assignee: TECHNISCHE UNIVERSITAET MUENCHEN, Munich (DE)

(21) Appl. No.: 17/757,536

(22) PCT Filed: Dec. 18, 2020
(86) PCT No.: PCT/EP2020/086972

§ 371 Date: Jun. 16, 2022

(30) Foreign Application Priority Data
Dec. 19, 2019 (EP) 19217921.6

(51) Int. Cl. A61L 27/34; A61L 27/54; A61L 29/08; A61L 29/16; A61L 31/10; A61L 31/16

(52) U.S. Cl. CPC A61L 27/34; A61L 27/54; A61L 29/085; A61L 29/16; A61L 31/10; A61L 31/16; A61L 2300/406; A61L 2300/41; A61L 2300/412

(57) ABSTRACT

The present invention relates to a method for preparation of a functionalized surface comprising the steps: a) coating of a carrier with a least one polymer selected from a polyanionic or polycationic polymer, b) addition of at least one compound to the coated carrier of step a), c) exposing the at least one polyanionic or polycationic polymer on the coated carrier of step b) to an organic solvent, resulting in compaction of the at least one polyanionic or polycationic polymer and thereby encapsulating the at least one compound, d) reversible cross-linking of the at least one polyanionic or polycationic polymer of step c) with at least one cross-linker; e) removal of the organic solvent. Furthermore, the invention relates to a functionalized surface, a functionalized surface for use in medicine and a method for releasing a compound ex vivo.

### TECHNICAL FIELD OF THE INVENTION

The present invention relates to a method for preparation of a functionalized surface comprising the steps: a) coating of a carrier with a least one polymer selected from a polyanionic or polycationic polymer, b) addition of at least one compound to the coated carrier of step a), c) exposing the at least one polyanionic or polycationic polymer on the coated carrier of step b) to an organic solvent, resulting in compaction of the at least one polyanionic or polycationic polymer and thereby encapsulating the at least one compound, d) reversible cross-linking of the at least one polyanionic or polycationic polymer of step c) with at least one cross-linker; e) removal of the organic solvent. Furthermore, the invention relates to a functionalized surface, a functionalized surface for use in medicine and a method for releasing a compound ex vivo.

### BACKGROUND ART

Post-surgery inflammations after implantation are one of the most common problems in modern clinical practice. The consequences for the patients reach from simple inconvenience to revision surgeries or even death. If the implant has to be removed as a consequence of inflammation, the duration of both, patient hospitalization and physical inactivity are increased. This leads to elevated economic and social expenses.

In conventional approaches, local antibiotic depots, e.g., antibiotic loaded bone cement, are used to reduce inflammatory reactions after implantation. However, these approaches are prone to induce local overdosing and also fail to provide ongoing inflammatory protection during the critical time window. Thus, a repeated administration of antibiotics becomes necessary, which in return often leads to antibiotic resistance of the pathogens.

As one further approach, antibiotic loaded bone cement have been developed as an implant material⁷; however, here the release mechanism is not very efficient since most of the antibiotics remain inside the cement instead of being released into the surrounding tissue⁸. Thus, control over the local amount of released drug cannot be fully reached.

WO2007/092179 discloses a device with nanocomposite coating for controlled drug release. The nanocomposite coating includes a matrix, a bioactive agent, and inorganic particles. The inorganic particles respond to a stimulus, preferably by generating heat. The stimulus may be a magnetic field or electromagnetic radiation. The response of the particles to the stimulus causes the matrix of the nanocomposite coating to undergo a volume change, for example, contracting or swelling, thereby releasing at least a portion of the bioactive agent.

WO 96/39949 discloses a method for triggering the release of a drug from a hydrogel polymer to tissue at a desired location of the body using a catheter. A portion of the catheter is coated on its outer surface with a polymer having the capacity to incorporate a predetermined substantial amount of drug which is immobilized in the polymer until released by triggering agent or condition that is different from physiological conditions. Upon contact with a triggering agent or condition, the polymer reacts, e.g., swells or contracts such that the drug is delivered to the desired body tissue.

Builders, Philip F.; European Journal of Pharmaceutics and Biopharmaceutics 72 (2009), 34-41 discloses mucinated cellulose microparticles for therapeutic and drug delivery purposes. It is thought that there is a pH-dependent responsiveness to swelling for mucin and mucinated cellulose microparticles.

In addition to advancements in site-specific implant design, inflammation is still a drawback associated with all kinds of implantations, which should be immediately overcome to prevent bacterial infection at the implant surface. Bacteria can reach the implant via the hematogenous route, during the operation, or they can spread from an external source of propagation from soft tissue infection. Bacterial adhesion, ending with biofilm formation, is a serious problem, which threatens the success of the operation, and it may even cause necrosis of the surrounding tissue¹′². Biomaterial-associated biofilm formation and tissue infection have been increasingly identified as one of the most frequent reasons for the failure of implanted medical devices (e.g., catheters, stents, and mechanical heart valves)³. Furthermore, infection followed by patient morbidity has been reported for 5% of all hip and joint replacement operations⁴. If bacterial colonization on an implant cannot be overcome, it can cause the formation of fibrous tissue around the implant. Here, the human body forms an interface between the healthy tissue and the implant, which causes poor patient compliance and reduced tissue integration.

Typically, antibiotic treatment is applied via oral administration or high concentration injection to prevent possible post-implantation inflammations. In oral systems, the possibility that a drug cannot be delivered to the target site at therapeutic concentrations is a serious issue. Therefore, to reach a therapeutic level through oral administration, high doses of antibiotics have to be used. This, in turn, can induce antibiotic resistance⁵. In addition, the overuse of systematically applied drugs can lead to negative side effects such as nephrotoxicity; oral administration of drugs is particularly likely to yield such side effects as, here, plasma drug concentrations occur that are typically much higher than the minimum inhibitory concentration of bacteria, and the excess antibiotic induces side effects⁶.

Consequently, there is an ongoing need for controlled release vehicles that liberate the required drug at desired concentrations but also induce an appropriate host response in the human body. That is, in particular, the case for the application of antibiotics as exemplarily discussed above but it is analogously true for the application of other drugs.

### SUMMARY OF THE INVENTION

The invention is directed to a method for preparation of a functionalized surface, comprising the steps:

a) coating of a carrier with a least one polymer selected from a polyanionic or polycationic polymer;
b) addition of at least one compound to the coated carrier of step a)
c) exposing the at least one polyanionic or polycationic polymer on the coated carrier of step b) to an organic solvent, resulting in compaction of the at least one polyanionic or polycationic polymer and thereby encapsulating the at least one compound,
d) reversible cross-linking of the at least one polyanionic or polycationic polymer of step c) with at least one cross-linker;
e) removal of the organic solvent.

In a second aspect, the invention is directed to a functionalized surface comprising:

a) a carrier
b) a coating on said carrier comprising
i) at least one compacted polymer selected from a polyanionic or polycationic polymer;
ii) at least one compound encapsulated by the compacted polymer and
iii) at least one reversible cross-linker.

In a third aspect, the invention is directed to a functionalized surface prepared according to the method above or the functionalized surface, as defined as second aspect of the invention above, wherein the compound is a pharmaceutically active compound, for use in medicine.

In a fourth aspect, the invention is directed to a functionalized surface prepared according to the method above or the functionalized surface, as defined as second aspect of the invention above, wherein the compound is a pharmaceutically active compound, for use in treatment of bacterial infections, tissue inflammation, or to stimulate tissue regeneration.

In a fifth aspect, the invention is directed to a method for releasing a compound ex vivo comprising exposing the functionalized surface prepared according to the method as defined above or the functional surface, as defined above, to a medium comprising a physiologically acceptable sodium chloride concentration, preferably a concentration of 90 to 500 mM, more preferably concentration of 100 to 200 mM, most preferably a concentration of 130 to 160 mM.

The present invention provides, a functionalized surface, coated with polyanionic or polycationic polymers which allows controlled release of compounds with high efficiency (up to >90%), including pharmaceutically active compounds. The invention allows release of a variety of different compounds, differing in types of chemical structure (polymeric, small molecule), net charge (anionic, cationic, neutral), and size, as demonstrated in example 1 and 7. The release of the compound is triggered by physiological conditions that remove the physiologically acceptable cross-linker. While for example Ca²⁺ may be used as reversible cross-linker, the release of Ca²⁺ in organic tissue is less desirable since Ca²⁺ may interfere in natural cell signaling. It has been found as part of the present invention that other, less harmful ions, such as Mg²⁺ are likewise suitable for this purpose (see examples 1 to 4). The present invention may be beneficial for coatings of medical implants to overcome post-surgery inflammations and bacterial colonization on the implant surfaces. In one embodiment, mucin may be used as a polyanionic polymer that can offer additional beneficial properties such as improved lubricity and anti-biofouling properties, which will remain after release of the compound, in particular, of a pharmaceutically active compound.

### BRIEF DESCRIPTION OF THE FIGURES

FIG. 1: Tetracycline standard curve. A TCL standard curve is prepared by measuring the absorbance values of serially diluted TCL solutions at 360 nm. This wavelength was determined by scanning a TCL solution in a wavelength range of 250-400 nm (inset).

FIG. 2: Vancomycin standard curve. A VAC standard curve is prepared by measuring the absorbance values of serially diluted VAC solutions at 282 nm. This wavelength was determined by scanning a VAC solution in a wavelength range of 250-330 nm (inset).

FIG. 3: Physiologically triggered drug release mechanism from a surface-bound mucin layer. For all groups, Mg²⁺ stabilized TCL (a) and VAN (b) loaded, or Fe²⁺ (c) and Zn²⁺ (d) stabilized TCL loaded mucin layers, it has been observed a release from the compacted mucin layer as soon as the system is exposed to a physiological trigger (for all data sets shown, physiological NaCl concentrations, i.e., 150 mM, are added at time point 0 for the data shown as full bars; for the data shown as open bars, the time point of NaCl addition is indicated by the dashed lines). The error bars represent the standard deviation as obtained from n=3 independent samples.

FIG. 4: Physiologically triggered release mechanism from surface-bound CM-dextran (polyanionic). It has been observed a release of TCL from the compacted dextran layer as soon as the system was exposed to a physiological trigger. The error bars represent the standard deviation as obtained from n=3 independent samples.

FIG. 5: Physiologically triggered release mechanism from surface-bound chitosan (polycationic). It has been observed a release of TCL from the compacted chitosan layer as soon as the system was exposed to a physiological trigger. The error bars represent the standard deviation as obtained from n=3 independent samples.

FIG. 6: Physiologically triggered release mechanism from surface-bound PLL (polycationic). It has been observed a release of TCL from the compacted PLL layer as soon as the system was exposed to a physiological trigger. The error bars represent the standard deviation as obtained from n=3 independent samples.

FIG. 7: Physiologically triggered drug release mechanism from a surface bound mucin layer. For all groups, a release of the model drug from the condensed mucin layer has been observed as soon as the system is exposed to a physiological trigger (for all data sets shown, physiological NaCl concentrations, i.e., 150 mM, are added after time point 0 for the data shown as black symbols; for the data shown as open symbols, no NaCl was added).

### DETAILED DESCRIPTION OF THE INVENTION

The solution of the present invention is described in the following, exemplified in the appended examples, illustrated in the Figures and reflected in the claims. □

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably. □

The invention is directed to a method for preparation of a functionalized surface.

The method comprises the steps:

Step a) Coating of a carrier with a least one polymer selected from a polyanionic or polycationic polymer.

Basically, the person skilled in the art may use any suitable coating process in order to coat a carrier with at least one polymer selected from a polyanionic or polycationic polymer. The coating process is dependent from the carrier used and the intended coating. Examplary coating processes are described in the example section.

The carrier is preferably made of material comprising silicon, polydimethylsiloxane (PDMS), polyethylene (PE), polyvinyl chloride (PVC), polyurethane (PU), polyethylene terephthalate (PET), poly(methylmethacrylate) (PMMA), polypropylene (PP), ceramics, and/or metal, more preferably of silicon, polydimethylsiloxane (PDMS), polyethylene (PE), polyvinyl chloride (PVC), polyurethane (PU), polyethylene terephthalate (PET), poly(methylmethacrylate) (PMMA), and polypropylene (PP, most preferably polydimethylsiloxane (PDMS).

The carrier may be an implant which is coated in the inventive process. The implant may be for example an artificial cardiac valve, a catheter, a stent, an intraocular lense, artificial vessels, replacements for tendons & ligaments, tubings, total joint replacements, vascular grafts, a skin replacement, a cardiac pace makers, a finger joint prosthesis, a breast implant, a testicle implant, a gastritic bag, drains, contact lenses.

TABLE 1
Examples for carrier materials and their applications in medical products, which
may benefit from the inventive coating resulting in the inventive functionalized surface

| Name | Monomer Structure | Medical Application |
|---|---|---|
| Polymethyl- methacrylate (PMMA) | 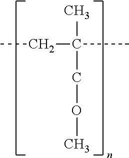 | tooth fillings & replacements, intra-ocular lenses, bone cement ^{14,15} |
| Polyethylene- terephthalate (PET) | 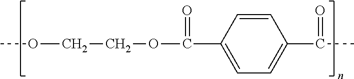 | artificial vessels, replacements for tendons & ligaments, surgical suture material ^{14,16} |
| Polyvinyl- carbonate (PVC) | 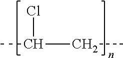 | tubings, catheters, blood pouches ^{14,17} |
| Polyethylene (PE) | 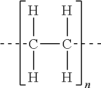 | medical containers, catheters, artificial tendons, total joint replacements ^{14,16,5} |
| Polyurethane (PU) | 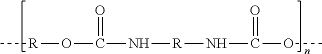 | artificial cardiac valves, stent coatings, balloon catheters, tubings, vascular grafts, skin replacement, cardiac pace makers, long term implants ^{14,18} |
| Polypropylene (PP) | 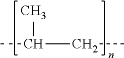 | finger joint prosthesis, grafts, non- degradable sutures ^{16,18} |
| Polydimethyl- siloxane (PDMS) | 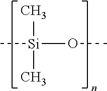 | flexible and small joint replacements, breast implants, testicle implants, transfusion and catheter tubings, gastric bags, drains, endoscopic windows, bandages, contact lenses ^{14,18} |

The at least one polymer may be a polyanionic polymer, preferably selected from mucin, CM-dextran, carboxymethyl cellulose, alginic acid, more preferably from mucin.

In one embodiment, the at least polymer is a polyanionic polymer, selected from CM-dextran, carboxymethyl cellulose, and alginic acid.

Mucins are a group of large glycoproteins with molecular weights up to a few MDa. Preferably mucins are used with a molecular weight of 500 kDa to 50 MDa (depending on the degree of polymerization), more preferably oligomeric mucin with a molecular range of 1-5 MDa.

The at least one polymer may be a polycationic polymer, preferably selected from chitosan, Poly-L-Lysine, or DEAE-dextran, more preferably chitosan, or Poly-L-Lysine.

Step b) Addition of at least one compound to the coated carrier of step a).

Basically, the compound may be any compound which shall be delivered with the inventive method. The compound may be anionic, cationic or neutral. In one embodiment the compound is anionic or cationic. Preferably, the compound is an organic compound. The compound may be a water soluble drug which preferably has a solubility in water of at least 0.2 μg/ml, more preferably at least 10 μg/ml, particular preferred at least 0.1 mg/ml. The compound may have a molecular weight of 50 to 12000 Da, preferably 200 to 10000 Da, more preferably 300 to 6000 Da.

The polycationic or polyanionic polymer may exhibit the same or a different overall charge than the compound added in step b). Preferably, the compound is a pharmaceutically active compound. More preferably, the compound is an antibiotic. Most preferably the compound is selected from Vancomycin, or Tetracycline, Chloramphenicol, Doxorubicin, CM Dextran, Dextran, DEAE Dextran, Atto 488 amine, Atto 488 carboxy (CAS Number: 923585-42-6), Atto 532 amine or a solvate, hydrate, salt, complex, racemic mixture, diastereomer, enantiomer, tautomer, and isotopically enriched forms thereof.

In one embodiment, the compound is selected from Vancomycin, Tetracycline, Chloramphenicol and Chloramphenicol.

Step c) Exposing the at least one polyanionic or polycationic polymer on the coated carrier of step b) to an organic solvent, resulting in compaction of the at least one polyanionic or polycationic polymer and thereby encapsulating the at least one compound.

The solvent is a protic or aprotic solvent. Preferably, the at least one polyanionic or polycationic polymer has low solubility in the solvent.

The solvent may be an alcohol. Preferably the alcohol comprises 1 to 3 hydroxyl groups, most preferably 3 hydroxyl groups. Preferably, the alcohol comprises 1 to 6 carbon atoms, more preferably 3 carbon atoms. Preferably, the alcohol is selected from glycerol, methanol, ethanol, propanol, butanol, pentanol, more preferably from glycerol and ethanol, most preferably from glycerol.

The solvent may be a linear or cyclic hydrocarbon, a linear or cyclic ether, or a linear or cyclic ester comprising 1 to 8 carbon atoms. Preferably, the linear or cyclic hydrocarbon, linear or cyclic ether, or a linear or cyclic ester is selected from pentane, hexane, heptane, octane, benzene, toluene, dichloromethane, ethyl acetate, tetrahydrofuran, diethyl ether, more preferably hexane.

Whilst not being bound by theory, it is thought that upon exposure of the at least polyanionic or polycationic polymer to an organic solvent, wherein the respective polymer preferably has a low solubility in the organic solvent, the respective polymer rearranges into the most thermodynamically favorable state. During the exposure to the organic solvent that is a compacted, particle-like state in order to minimize the surface and the interaction with the solvent. During this rearrangement to the thermodynamic more favorable state during exposure to the solvent, the compound of step b) is encapsulated.

Step d) reversible cross-linking of the at least one polyanionic or polycationic polymer of step c) with at least one cross-linker.

It has been found by the inventors, that the compacted, particle-like state of step c) may be reversibly stabilized by adding at least one cross-linker.

The term “reversible” means that under certain trigger conditions the cross-linker may be removed and the at least one polyanionic or the at least one polycationic polymer may return or refold to the uncompacted state, then releasing the encapsulated compound.

Preferably, the cross-linker is an ion. More preferably a monovalent, divalent or trivalent ion, most preferably a divalent or trivalent ion, particularly preferred a divalent ion

Preferably, the cross-linker is a cation if the polymer is a polyanionic polymer and an anion if the polymer is a polycationic polymer.

If the at least one crosslinker is a divalent cation, the at least one cross-linker is preferably selected from Ba²⁺,′Ca²⁺, Mg²⁺, Fe²⁺, Zn²⁺, more preferably from Mg²⁺, Fe²⁺, Zn²⁺, most preferably Mg²⁺.

If the at least one cross-linker is a divalent anion, the at least one cross-linker is preferably selected from SO₄²⁻, PO₄²⁻, more preferably selected from SO₄²⁻.

Step e) removal of the organic solvent.

It has been found by the inventors that after cross-linking, the organic solvent may be removed while the polyanionic or polycationic polymer stays in the compacted state.

Step e) may comprise replacing the organic solvent by ultrapure water, wherein the ultra-pure water preferably has an ion content of lower than 1 μg/L more preferred lower than 0.5 μg/L, most preferred lower than 0.05 μg/L.

It has been found that the release of the compound is not triggered by ultrapure water.

The invention is further directed to a functionalized surface.

The functionalized surface comprises a) a carrier as defined above.

Furthermore, the functionalized surface comprises b) a coating on said surface.

The coating comprises i) at least one compacted polymer selected from a polyanionic or polycationic polymer as defined above.

The coating comprises ii) at least one compound encapsulated by the compacted polymer as defined above.

The coating comprises iii) at least one reversible cross-linker as defined above.

The invention is further directed to a functionalized surface prepared according to the method as described above or to a functionalized surface as described above, wherein the compound is a pharmaceutically active compound, for use in medicine.

The invention is further directed to a functionalized surface prepared according to the method as described above or to a functionalized surface as described above, wherein the compound is a pharmaceutically active compound, preferably an antibiotic, for use in treatment of bacterial infections, tissue inflammation, or to stimulate tissue regeneration.

, The use in medicine and the use in treatment preferably comprises exposing the functionalized surface to a medium comprising preferably a physiologically acceptable sodium chloride concentration, more preferably a concentration of 90 to 500 mM, most preferably concentration of 100 to 200 mM, particular preferred a concentration of 130 to 160 mM. Exposure to a physiologically acceptable sodium chloride concentration triggers the release of the pharmaceutically active compound.

The invention is further directed to a method for releasing a compound ex vivo comprising exposing the functionalized surface prepared according to the method, as described above, or the functional surface, as described above, to a medium comprising preferably a physiologically acceptable sodium chloride concentration, more preferably a concentration of 90 to 500 mM, most preferably concentration of 100 to 200 mM, particular preferred a concentration of 130 to 160 mM. Exposure to a physiologically acceptable sodium chloride concentration triggers the release of the compound.

A better understanding of the present invention and of its advantages will be had from the following examples, offered for illustrative purposes only. The examples are not intended to limit the scope of the present invention in any way.

### EXAMPLES OF THE INVENTION

In the following it is shown that differently charged macromolecules can be grafted onto a silicone model substrate, loaded with drugs, condensed with glycerol, stabilized using oppositely charged ions and finally forced to release their cargo in the presence of a physiological trigger. As an overview, in Table 2, different conditions are listed which have been tested. Detailed experimental protocols and results can be found in the following disclosure.

TABLE 2
Overview over the tested coatings, ions, and drugs

| Coating | Charge | Stabilizing ion | Loaded drug |
|---|---|---|---|
| mucin | polyanionic | Mg²⁺ | TCL (anionic) |
| | | Mg²⁺ | VAN (cationic) |
| | | Fe²⁺ | TCL |
| | | Zn²⁺ | TCL |
| CM dextran | polyanionic | Mg²⁺ | TCL |
| chitosan | polycationic | SO₄²⁻ | TCL |
| Poly-L-lysine | polycationic | SO₄²⁻ | TCL |

### Example 1: Drug Release from Surface-Bound Mucin Layers

1.1 Mucin Purification

Porcine gastric mucin MUC5AC was purified manually as described previously¹⁰. In brief, mucus was obtained from gently rinsed pig stomachs by manual scraping the surface of the gastric tissue. The collected mucus was diluted 5-fold in 10 mM sodium phosphate buffer (pH=7.0) containing 170 mM NaCl and 0.04% Sodium azide (Carl Roth, Karlsruhe, Germany) and stirred at 4° C. overnight. Cellular debris was removed via two centrifugation steps (first run: 8300 g at 4° C. for 30 min; second run: 15000 g at 4° C. for 45 min) and a final ultracentrifugation step (150000 g at 4° C. for 1 h). Subsequently, the mucins were separated from other macromolecules by size exclusion chromatography using an ÄKTA purifier system (GE Healthcare, Munich, Germany) and an XK50/100 column packed with Sepharose 6FF. The obtained mucin fractions were pooled, dialyzed against ultrapure water and concentrated by cross-flow filtration. The concentrate was then lyophilized and stored at −80° C. until further use.

1.2 Coating Process for MUC5AC

The coupling reaction was performed as described previously⁹. Briefly, PDMS was treated with O₂ plasma at 0.4 mbar pressure and an intensity of 30 W for 90 s. The plasma treatment replaces the methoxy groups on the polymer surface with hydroxyl groups, which enables a covalent attachment of silane molecules. The silane was used as a coupling agent to further allow for attaching porcine gastric mucin to the surface via carbodiimide coupling. Thus, we here used N-[3-Trimethoxysilyl)propyl]ethylenediamine triacetic acid trisodium salt (TMS-EDTA, abcr, Karlsruhe, Germany), which carries three carboxy groups on the other end. TMS-EDTA was diluted to a final concentration of 0.1% (w/v) in 10 mM Acetate buffer (pH=4.5). The activated PDMS samples were then incubated in the silane solution for 5 h at 60° C. Afterwards, the samples were washed in 80% ethanol (Carl Roth) for 1 h to remove unbound residues before they were placed in the oven at 60° C. for another 60 min to stabilize the bond between the PDMS and the silane.

In the next step, the carboxyl groups of the silane were activated. Therefore, the surface was covered with 100 mM MES buffer (pH=5.0) containing 5 mM 1-Ethyl-3-(3-dimethylaminopropyl)-carbodiimid-hydrochloride (EDC, Carl Roth) and 5 mM N-Hydroxysulfosuccinimide (sulfo-NHS, abcr) at RT for 30 min. Afterwards, the EDC-NHS solution was replaced by phosphate-buffered saline (pH=7.4, Lonza, Velviers, Belgium) containing 0.1% (w/v) of purified mucin and stored at 4° C. overnight. Amine groups from the mucin protein core then react with the EDC activated groups of the silane and form a stable covalent bond.

1.3 Release Experiments from Suface Bound Mucin

To determine the release of either Vancomycin hydrochloride (VAC, positively charged at neutral pH, Applichem, Darmstadt, Germany) or tetracycline hydrochloride (TCL, negatively charged at neutral pH, Applichem) from a mucin coated surface, first the bottom of polystyrene cuvettes (BrandTech™ macro, WVR, used for TCL release) or UV transparent cuvettes (WVR, for VAN release) was covered with PDMS. Therefore, a two-component commercial PDMS system (Sylgard 184, Dow Corning) was prepared by mixing prepolymer and crosslinker in a ratio of 10:1, degassing the mixture in a vacuum chamber for 1 h and subsequently pouring 300 μL into the cuvette (this amount is chosen such that the thickness of the PDMS layer does not interfere with the light of the UV spectrometer by blocking the light that is guided into the cuvette) using a displacement pipette. PDMS was allowed to crosslink at 60° C. overnight. Afterwards, this PDMS layer was coated with mucin as described in Section 1.2.

The mucin layer was loaded with the drug by incubating it in a drug-containing solution (TCL or VAC, 0.5 mg/mL) at 4° C. overnight. On the next day, the mucin layer was condensed by adding a 30% glycerol solution. Afterwards, 50 mM of either MgCl₂, FeCl₂ or ZnCl₂ was added to stabilize the condensed layer, and the system was allowed to incubate for 1 h. Before starting the release experiment, excessive glycerol, drug, and salt were removed and replaced by 2 mL of ultrapure water.

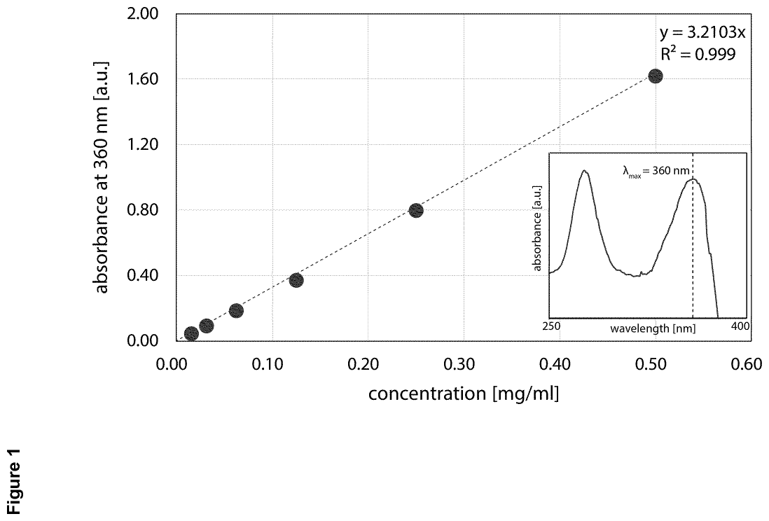

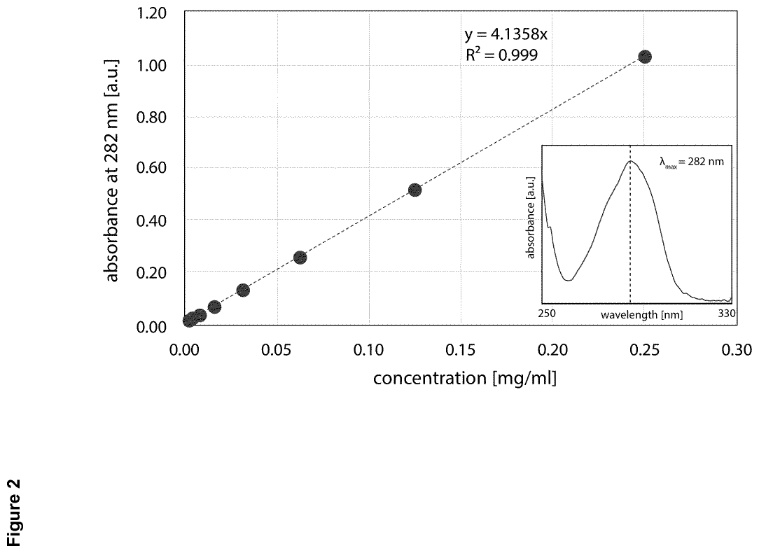

The drug release from the condensed mucin layer was initiated by replacing the ultrapure water with 150 mM of sodium chloride solution, and the release of TCL was tracked spectroscopically with a specord210 spectral photometer (Analytikjena, Jena, Germany) at 360 nm. The amount of released drug was determined by a standard curve which was generated by measuring serial dilutions of a drug solution (see FIG. 1). The release of VAN was also tracked spectroscopically, however, at 282 nm (for calibration curve, see FIG. 2).

1.4 Results

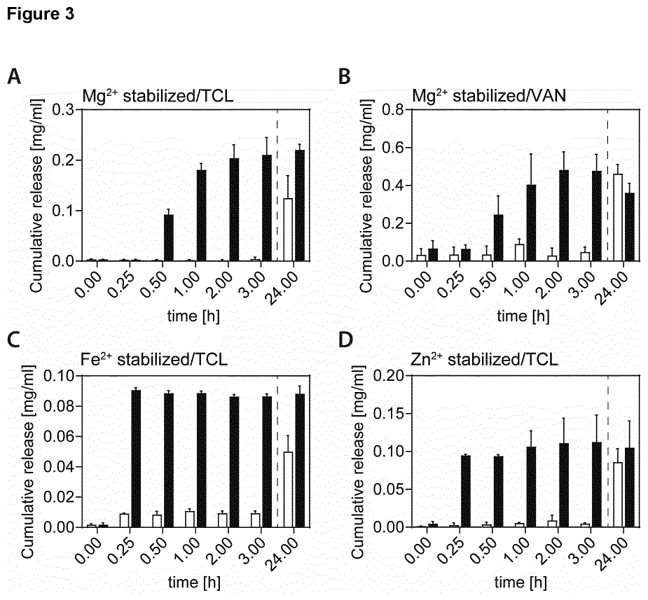

When the TCL-loaded, fully condensed mucin system was exposed to the physiological trigger solution, a release of the therapeutical agent has been observed after −30 min. After ˜2 h, the drug concentration in the fluid reached a plateau value (FIG. 3a, full bars).

If the same system is exposed to ultrapure water instead of a sodium chloride solution (this is supposed to simulate storage of the drug-loaded coating), the drug stays trapped in the mucin layer; however, it can also be released later by adding the trigger (FIG. 3a, open bars). Both other control groups (without either Mg²⁺ or glycerol condensation, see Table 3) show little to no release at all-even if the correct trigger is used (since here, the condensation step is missing, which is required for successful drug integration into the mucin polymer layer).

TABLE 3
Tetracycline release from condensed mucin samples stabilized with Mg²⁺ (n = 3).

| | Group 1 | Group 2 | Group 3 | Group 4 |
|---|---|---|---|---|
| condensation | Glycerol | glycerol | — | — |
| stabilization | Mg²⁺ | Mg²⁺ | Mg²⁺ | — |
| trigger | 150 mM NaCl | — | 150 mM NaCl | 150 mM NaCl |
| time [h] | C_{TCL} [mg/mL] | C_{TCL} [mg/mL] | C_{TCL} [mg/mL] | C_{TCL} [mg/mL] |
| 0 | 0.0034 ± 0.0003 | 0.0035 ± 0.0009 | 0.0072 ± 0.0036 | 0.0032 ± 0.0006 |
| 0.25 | 0.0029 ± 0.0006 | 0.0025 ± 0.0009 | 0.0069 ± 0.0043 | 0.0023 ± 0.0006 |
| 0.5 | 0.0920 ± 0.0110 | 0.0021 ± 0.0011 | 0.0095 ± 0.0060 | 0.0027 ± 0.0007 |
| 1.0 | 0.1805 ± 0.0131 | 0.0020 ± 0.0011 | 0.0175 ± 0.0187 | 0.0025 ± 0.0008 |
| 2.0 | 0.2040 ± 0.0266 | 0.0017 ± 0.0012 | 0.0181 ± 0.0191 | 0.0022 ± 0.0005 |
| 3.0 | 0.2104 ± 0.0345 | 0.0042 ± 0.0035 | 0.0174 ± 0.0192 | 0.0038 ± 0.0043 |

To assess the effect of the charge of the drug on the release process, we also tested a positively charged drug, Vancomycin hydrochloride (VAN), which is widely preferred for implantation operations to prevent post-operative infection.

Similarly, after exposing the system to the physiological trigger, VAN release was observed (FIG. 3b, full bars). In contrast, almost no release was observed for samples where the trigger was absent (FIG. 3b, open bars). Also here, we performed similar control measurements as described above, i.e. without Mg²⁺ or without glycerol condensation (see Table 4).

TABLE 4
Vancomycin release from condensed mucin samples stabilized with Mg²⁺ (n = 3).

| | Group 1 | Group 2 | Group 3 | Group 4 |
|---|---|---|---|---|
| condensation | Glycerol | glycerol | — | — |
| stabilization | Mg²⁺ | Mg²⁺ | Mg²⁺ | — |
| trigger | 150 mM NaCl | water | 150 mM NaCl | 150 mM NaCl |
| time [h] | C_{TCL} [mg/mL] | C_{TCL} [mg/mL] | C_{TCL} [mg/mL] | C_{TCL} [mg/mL] |
| 0 | 0.0014 ± 0.0010 | 0.0008 ± 0.0008 | 0.0018 ± 0.0008 | 0.0033 ± 0.0022 |
| 0.25 | 0.0013 ± 0.0006 | 0.0008 ± 0.0010 | 0.0022 ± 0.0008 | 0.0029 ± 0.0015 |
| 0.5 | 0.0051 ± 0.0029 | 0.0008 ± 0.0011 | 0.0024 ± 0.0010 | 0.0034 ± 0.0014 |
| 1.0 | 0.0083 ± 0.0042 | 0.0022 ± 0.0007 | 0.0034 ± 0.0009 | 0.0022 ± 0.0005 |
| 2.0 | 0.0098 ± 0.0031 | 0.0007 ± 0.0009 | 0.0034 ± 0.0003 | 0.0011 ± 0.0010 |
| 3.0 | 0.0097 ± 0.0029 | 0.0012 ± 0.0006 | 0.0034 ± 0.0003 | 0.0013 ± 0.0011 |

In a third step, magnesium was replaced as a stabilizing agent with either iron (FIG. 3c) or zinc (FIG. 3d). Also here, we observe a similar pattern, i.e. the drug is stored in the condensed mucin layer until the physiological trigger is introduced to the system.

### Example 2: Drug Release from Surface-Bound CM-Dextran (Polyanionic Polysaccharide)

2.1 Coating Process for Dextran

PDMS was prepared and activated with oxygen plasma as described above (see Section 1.2). Afterwards an amine-functionalized silane (3-Aminopropyltriethoxysilan, APTES, Sigma Aldrich) was diluted to 0.1% in 2-propanol and the cuvettes were incubated with this solution at 60° C. for 5 h. Afterwards, the samples were washed in 80% ethanol (Carl Roth) for 1 h to remove unbound residues before they were placed in the oven at 60° C. for another 60 min to stabilize the bond between the PDMS and the silane.

In parallel, carboxy modified dextran (150 kDa, TdB Consultancy AB, Uppsala Sweden) was dissolved to a concentration of 1% (w/v) in 10 mM MES buffer (pH=5) and 5 mM EDC and 5 mM sulfo-NHS were added to activate the carboxyl groups. The solution was incubated for 3 h at RT to make sure that unreacted EDC and NHS were hydrolyzed before the solution was diluted 1:10 in PBS (pH=7.4) to obtain a final dextran concentration of 0.1% (w/v). The solution was filled into the cuvettes and allowed to react at 4° C. overnight. Amine groups of the silane molecule then react with the EDC activated groups of the dextran and form a stable covalent bond.

2.2 Release Experiments from Surface-Bound Dextran

To determine the drug release of tetracycline hydrochloride (TCL, negatively charged at neutral pH, Applichem) from a CM dextran-coated surface, first, the bottom of polystyrene cuvettes (BrandTech™ macro, WVR) was covered with PDMS as described before (see Section 1.3). Afterwards, this PDMS layer was coated with chitosan as described in Section 2.1.

The dextran layer was loaded with the drug by incubating it in a drug-containing solution (TCL, 0.5 mg/mL) at 4° C. overnight. On the next day, the dextran layer was condensed by adding a 30% glycerol solution. Afterwards, 50 mM of MgCl₂ was added to stabilize the condensed layer, and the system was allowed to incubate for 1 h. Before starting the release experiment, excessive glycerol, drug, and salt were removed and replaced by 2 mL of ultrapure water.

The drug release from the condensed dextran layer was initiated by replacing the ultrapure water with 150 mM of sodium chloride solution, and the release of TCL was tracked spectroscopically with a specord210 spectral photometer (Analytikjena, Jena, Germany) at 360 nm. The amount of released drug was determined by a standard curve which was generated by measuring serial dilutions of a drug solution (see FIG. 1).

2.3 Results

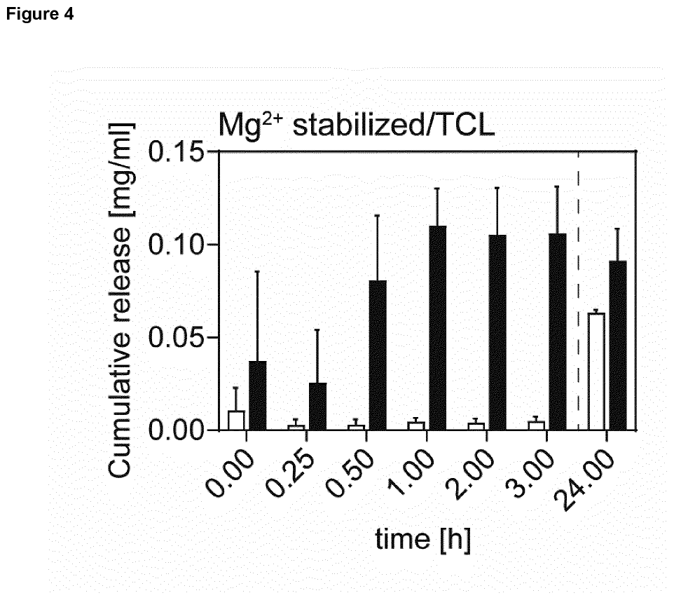

When the TCL-loaded, fully condensed dextran system was exposed to the physiological trigger solution, a release of the therapeutical agent has been observed. After −60 min, the drug concentration in the fluid reached a plateau value (FIG. 4, full bars). If the same system is exposed to ultrapure water instead of a sodium chloride solution (this is supposed to simulate storage of the drug-loaded coating), the drug stays trapped in the mucin layer; however, it can also be released later by adding the trigger (FIG. 4, open bars).

### Example 3: Drug Release from Surface-Bound Chitosan (Polycationic Polysaccharide)

3.1 Coating Process for Chitosan

The coupling reaction was performed as described for the coupling of porcine gastric mucin (see Section 1.2) except for the last step: After the carboxyl groups of the silane had been activated, the EDC-NHS solution was replaced by 2% acetic acid containing 0.1% (w/v) of chitosan (95/3000, Heppe Medical Chitosan GmbH, Halle, Germany) and stored overnight at 4° C. Amine groups of the chitosan molecule then react with the EDC activated groups of the silane and form a stable covalent bond.

3.2 Release Experiments from Surface-Bound Chitosan

To determine the drug release of tetracycline hydrochloride (TCL, negatively charged at neutral pH, Applichem) from a chitosan-coated surface, first, the bottom of polystyrene cuvettes (BrandTech™ macro, WVR) was covered with PDMS as described before (see Section 1.3). Afterwards, this PDMS layer was coated with chitosan as described in Section 3.1.

The chitosan layer was loaded with the drug by incubating it in a drug-containing solution (TCL, 0.5 mg/mL) at 4° C. overnight. On the next day, the chitosan layer was condensed by adding a 30% glycerol solution. Afterwards, 50 mM of MnSO₄ was added to stabilize the condensed layer, and the system was allowed to incubate for 1 h. Before starting the release experiment, excessive glycerol, drug, and salt were removed and replaced by 2 mL of ultrapure water.

The drug release from the condensed chitosan layer was initiated by replacing the ultrapure water with 150 mM of sodium chloride solution, and the release of TCL was tracked spectroscopically with a specord210 spectral photometer (Analytikjena, Jena, Germany) at 360 nm. The amount of released drug was determined by a standard curve which was generated by measuring serial dilutions of a drug solution (see FIG. 1).

3.3 Results

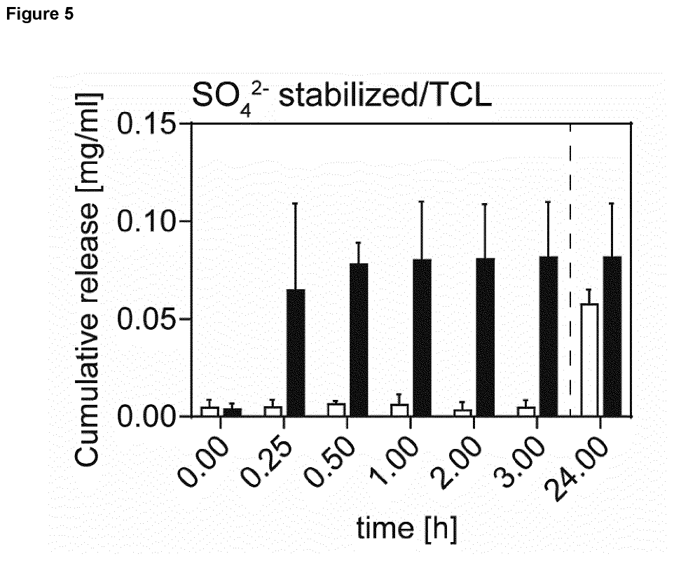

When the TCL-loaded, fully condensed chitosan system was exposed to the physiological trigger solution, a release of the therapeutical agent has been observed after ˜15 min. After ˜60 min, the drug concentration in the fluid reached a plateau value (FIG. 5, full bars). If the same system is exposed to ultrapure water instead of a sodium chloride solution (this is supposed to simulate storage of the drug-loaded coating), the drug stays trapped in the mucin layer; however, it can also be released later by adding the trigger (FIG. 5, open bars).

Both other control groups (without 50₄²⁻ or glycerol condensation, see Table 5) show little to no release at all-even if the correct trigger was used (since here, the condensation step is missing, which is required for successful drug integration into the chitosan polymer layer).

TABLE 5
Tetracycline release from SO₄²⁻ condensed samples (n = 3).

| | Group 1 | Group 2 | Group 3 | Group 4 |
|---|---|---|---|---|
| condensation | glycerol | glycerol | — | — |
| stabilization | SO₄²⁻ | SO₄²⁻ | SO₄²⁻ | — |
| trigger | 150 mM NaCl | water | 150 mM NaCl | 150 mM NaCl |
| time [h] | C_{TCL} [mg/mL] | C_{TCL} [mg/mL] | C_{TCL} [mg/mL] | C_{TCL} [mg/mL] |
| 0 | 0.0043 ± 0.0024 | 0.0052 ± 0.0035 | 0.0019 ± 0.0020 | 0.0026 ± 0.0015 |
| 0.25 | 0.0651 ± 0.0438 | 0.0052 ± 0.0033 | 0.0032 ± 0.0029 | 0.0031 ± 0.0029 |
| 0.5 | 0.0783 ± 0.0106 | 0.0068 ± 0.0011 | 0.0029 ± 0.0023 | 0.0034 ± 0.0024 |
| 1.0 | 0.0807 ± 0.0295 | 0.0065 ± 0.0050 | 0.0022 ± 0.0033 | 0.0045 ± 0.0035 |
| 2.0 | 0.0812 ± 0.0275 | 0.0037 ± 0.0037 | 0.0023 ± 0.0030 | 0.0038 ± 0.0022 |
| 3.0 | 0.0821 ± 0.0272 | 0.0051 ± 0.0033 | 0.0025 ± 0.0034 | 0.0037 ± 0.0019 |

### Example 4: Drug Release from Surface-Bound Poly-L-Lysine (PLL, Polycationic Polypeptide)

4.1 Coating Process for PLL

PDMS was prepared and activated with oxygen plasma as described above (see Section 1.2). Afterwards an azide modified surface for “click”-chemistry was generated by covalently bonding 6-azidosulfonyl-hexyltriethoxysilane (ASH-TES, abcr GmbH) to the activated surface. ASH-TES was diluted to 0.13% (v/v) in methanol (MeOH, >99.9%, Carl Roth) and acetate buffer (pH=4.5) was slowly added to obtain a 3:1 (MeOH:HOAc) solution with a final ASH-TES concentration of 0.1% (v/v). The ASH-TES solution is transferred into the cuvettes and allowed to react at 60° C. for 5 h. Afterwards, the samples were washed in 80% ethanol (Carl Roth) for 1 h to remove unbound residues before they were placed in the oven at 60° C. for another 60 min to stabilize the bond between the PDMS and the silane.

Here, an end-functionalized alkynyl-poly(L-lysine hydrobromide) molecule (aPLL, Alamanda Polymers), is used. To covalently attach aPLL to the PDMS surface, 1,3-dipolar cycloaddition is used, which has emerged as one of the most popular methods to employ the principle of “click”-chemistry5l. Although the 1,3-dipolar cycloaddition typically requires elevated temperatures, a rather long reaction time, and provides poor selectivity towards the reaction products, these restrictions have been remedied by employing a copper-(I)-catalyzed reaction scheme (CuAAC)¹⁰. In this modified form, the reaction is especially useful for bioconjugation at RT¹¹.

Here, a mixture of 1,4-diazabicyclo-[2,2,2]-octane (DABCO), glacial acetic acid (AcOH), and Cu(I) ions is prepared according to Sarode et al.¹³. Therefore, 0.03 mmol copper sulfate pentahydrate (CuSO4×5H₂O), 0.12 mmol sodium ascorbate (NaAsc), and 0.06 mmol 1,4-diazabicyclo[2.2.2]octane (DABCO, Carl Roth) were dissolved in 2 mL ultrapure water. Then, 0.06 mmol AcOH was added to the mixture and 5 mg aPLL were added to this solution to obtain an aPLL concentration of 0.1%. As before, the reaction was allowed to take place at 4° C. overnight.

4.2 Release Experiments from Surface-Bound PLL

To determine the drug release of tetracycline hydrochloride (TCL, negatively charged at neutral pH, Applichem) from a PLL coated surface, first, the bottom of polystyrene cuvettes (BrandTech™ macro, WVR) was covered with PDMS as described before (see Section 1.3). Afterwards, this PDMS layer was coated with PLL as described in Section 4.1.

The PLL layer was loaded with the drug by incubating it in a drug-containing solution (TCL, 0.5 mg/mL) at 4° C. overnight. On the next day, the PLL layer was condensed by adding a 30% glycerol solution. Afterwards, 50 mM of MnSO₄ was added to stabilize the condensed layer, and the system was allowed to incubate for 1 h. Before starting the release experiment, excessive glycerol, drug, and salt were removed and replaced by 2 mL of ultrapure water.

The drug release from the condensed PLL layer was initiated by replacing the ultrapure water with 150 mM of sodium chloride solution, and the release of TCL was tracked spectroscopically with a specord210 spectral photometer (Analytikjena, Jena, Germany) at 360 nm. The amount of released drug was determined by a standard curve which was generated by measuring serial dilutions of a drug solution (see FIG. 1).

4.3 Results

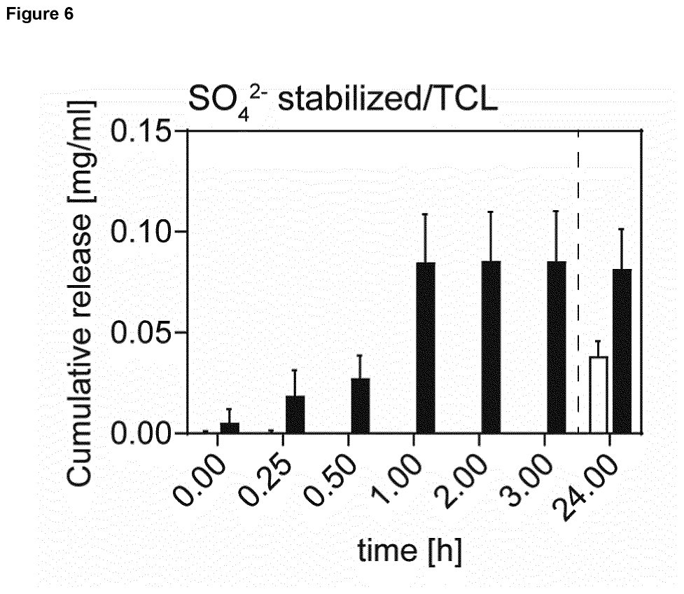

When the TCL-loaded, fully condensed PLL system was exposed to the physiological trigger solution, a release of the therapeutical agent after −15 min has been observed. After −60 min, the drug concentration in the fluid reached a plateau value (FIG. 6, full bars). If the same system is exposed to ultrapure water instead of a sodium chloride solution (this is supposed to simulate storage of the drug-loaded coating), the drug stays trapped in the mucin layer; however, it can also be released later by adding the trigger (FIG. 6, open bars).

### Example 5: Testing of Different Organic Solvents in Step c)

TABLE 6
Testing of different organic solvents in step c)

| | Peak 1 | | Peak 2 | | Peak 3 | | |
|---|---|---|---|---|---|---|---|
| Organic | | Size | | Size | | Size | |
| solvent | % | [nm] | % | [nm] | % | [nm] | PDI |
| Glycerol | 96 | 281 ± 60 | 4 | 55 ± 37 | | | 0.25 |
| Ethanol | 67 | 360 ± 105 | 23 | 497 ± 765 | 10 | 2421 ± 2068 | 0.55 |
| Hexane | 66 | 508 ± 295 | 21 | 521 ± 762 | 3 | 1503 ± 2581 | 0.65 |

When mucin molecules are compacted in the presence of glycerol and 50 mM Mg²⁺, they form compacted particles with uniform hydrodynamic sizes (PDI=0.25) as proven by Dynamic Light Scattering (DLS) measurements (see Table 6). This strategy is used to compact surface-bound mucin and the other polymers as well, in order to entrap compounds.

When glycerol is replaced with ethanol or hexane, mucin solutions still can form compacted structures (˜65% showed similar size distributions). However, the rest of the molecules formed large agglomerates and heterogeneous size distributions (PDI=0.55-0.65), indicating mucin could be partially compacted.

The data shows that ethanol and hexane could serve as alternatives to glycerol.

### Example 5: Encapsulating Chemically and Structurally Different Drugs of Different Molecular Weight and Net Charge

As model drugs for additional release experiments different molecules as summarized in Table 7 have been chosen:

TABLE 7
Model drugs overview. The table depicts name, abbreviation, net charge and
molecular weight of the 8 different model drugs as tested here. Furthermore,
it shows the concentration of the solution used for the drug loading process
as well as the wavelength at which absorption is detected.

| | | Net charge | Molecular | | Concentration | | |
|---|---|---|---|---|---|---|---|
| Name | Abbreviation | (at pH 7) | Weight | | (in H₂O) | | Absorbance |
| Chloramphenicol | CHL | neutral | 323 | Da | 0.5 | mg/mL | 320 nm |
| Doxorubicin | DOX | cationic | 543 | Da | 25 | μg/mL | 480 nm |
| CM Dextran | Dex (−) | anionic | 4000 | kDa | 0.5 | mg/mL | 495 nm |
| Dextran | Dex (0) | neutral | 4000 | kDa | 0.5 | mg/mL | |
| DEAE Dextran | Dex (+) | cationic | 3000-6000 | kDa | 0.5 | mg/mL | |
| Atto 488 carboxy | Atto (−) | anionic | 804 | Da | 0.5 | μg/mL | 505 nm |
| Atto 532 amine | Atto (0) | neutral | 916 | Da | 0.5 | μg/mL | 535 nm |
| Atto 488 amine | Atto (+) | cationic | 904 | Da | 0.5 | μg/mL | 505 nm |

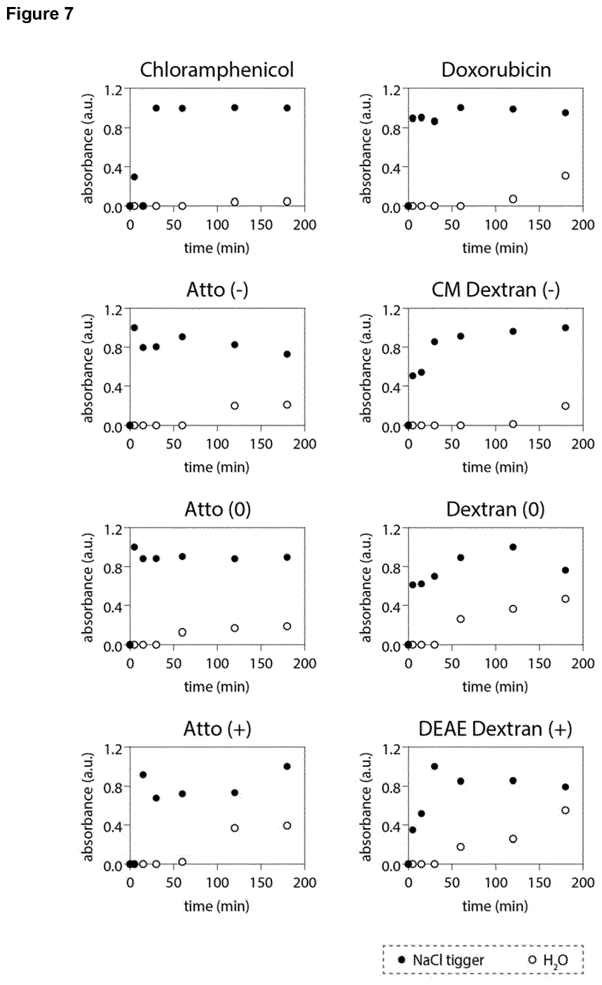

In order to determine the release of those model drugs from a mucin coated surface, first the bottom of polystyrene cuvettes (BrandTech™ macro, WVR, used for TCL release) was covered with PDMS and then coated with mucin as described before. Then mucin layer was loaded with the model drug by incubating it in a drug containing solution (see Table 7 for concentrations) at 4° C. overnight. On the next day, the mucin layer was condensed by adding a 30% glycerol solution. Afterwards, 50 mM of MgCl₂ was added to stabilize the condensed layer, and the system was allowed to incubate for 1 h. Before starting the release experiment, excessive glycerol, drug, and salt were removed and replaced by 2 mL of ultrapure water. The drug release from the condensed mucin layer was initiated by replacing the ultrapure water with 150 mM of sodium chloride solution, and the release of TCL was tracked spectroscopically with a specord210 spectral photometer (Analytikjena, Jena, Germany) at the corresponding wavelength for each molecule (see Table 7 for details). For illustration the absorbance values were normalized to the highest occurring absorbance value for each molecule. For all molecules tested here an immediate release has been observed as soon as the condensed and stabilized mucin layer is exposed to the salt trigger (FIG. 7, black symbols in each graph). For the control groups, i.e. where no salt was added, only little uncontrolled release can be observed (FIG. 7, open symbols in each graph).

### REFERENCES

1. Hogan, A., Heppert, V. G. & Suda, A. J. Osteomyelitis. Achives of Orthopaedic and Trauma Surgery 133, 1183-1196, (2013).
2. Vasso, M. Schiavone Panni, A., De Martino, I. & Gasparini, G. Prosthetic knee infection by resistant bacteria: the worst-case scenario. Knee Surgery, Sports Traumatology Athroscopy 24, 3140-3146, (2016).
3. Mahmoudi, M. et al. Infection-resistent MRI-visible scaffolds for tissue engineering applications. Bioimpacts 6, 111-115, (2016).
4. B., A., D., H. & A., H. Antibiotiic prophylaxis for wound infections in total joint arthoplasty. The Journal of Bone and Joint Surgery. British volume 90-B, 915-919, (2008).
5. Gao, P., Nie, X., Zou, M., Shi, Y. & Cheng, G. Recent advances in materials for extended-release antibiotic delivery system. The Journal of Antibiotics 64, 625 (2011).
6. Hammett-Stabler, C. A. & Johns, T. Laboratory guidelines for monitoring of antimicrobial drugs. Clinical Chemistry 44, 1129-1140 (1998).
7. Passuti, N. & Gouin, F. Antibiotic-loaded bone cement in orthopedic surgery. Joint Bone Spine 70, 169-174 (2003).
8. Hendriks, J. G. E., van Horn, J. R., van der Mei, H. C. & Busscher, H. J. Backgrounds of antibiotic-loaded bone cement and prosthesis-related infection. Biomaterials 25, 545-556 (2004).
9. Winkeljann, B. Leipold, P. M. A. & Lieleg, O. Macromolecular coatings enhance the tribological performance of polymer-based lubricants. Advanced Materials Interfaces 6, (2019)
10. Schömig, V. J. et al. An optimized purification process for porcine gastric mucin with preservation of its native functional properties. RSC Advances 6, 44932-44943 (2016).
11. Bock, V. D., Hiemstra, H. & van Maarseveen, J. H. Cul-Catalyzed Alkyne-Azide “Click” Cycloadditions from a Mechanistic and Synthetic Perspective. European Journal of Organic Chemistry 2006, 51-68 (2006).
12. Meldal, M. & Tornøe, C. W. Cu-Catalyzed Azide-Alkyne Cycloaddition. Chemical Reviews 108, 2952-3015 (2008).
13. Sarode, P. B., Bahekar, S. B. & Chandak, H. S. DABCO/AcOH Jointly Accelerated Copper(I)-Catalysed Cycloaddition of Azides and Alkynes on Water at Room Temperature. Synlett 27, 2681-2684 (2016).
14. Wintermantel, E., Ha, S. Medizintechnik. 1379 (Springer-Verlag Berlin Heidelberg, 2009).
15. Kaiser, W. Kunststoffchemie für Ingenieure. (Hanser, 2006).
16. Abts, G. Kunststoff-Wissen für Einsteiger. (Carl Hanser Verlag GmbH Co KG, 2016).
17. Hellerich, W. & Harsch, G. Werkstoff-Führer Kunststoffe.
18. Puoci, F. Advanced polymers in medicine. (Springer, 2015).

1. A method for preparation of a functionalized surface, comprising the steps:
a) coating of a carrier with a least one polymer selected from a polyanionic or polycationic polymer;
b) addition of at least one compound to the coated carrier of step a);
c) exposing the at least one polyanionic or polycationic polymer on the coated carrier of step b) to an organic solvent, resulting in compaction of the at least one polyanionic or polycationic polymer and thereby encapsulating the at least one compound;
d) reversible cross-linking of the at least one polyanionic or polycationic polymer of step c) with at least one cross-linker;
e) removal of the organic solvent.

2. A functionalized surface comprising:
a) a carrier
b) a coating on said carrier comprising
i) at least one compacted polymer selected from a polyanionic or polycationic polymer;
ii) at least one compound encapsulated by the compacted polymer and
iii) at least one reversible cross-linker.

3. A functionalized surface prepared according to the method of claim 1 or the functionalized surface according to claim 2, wherein the compound is a pharmaceutically active compound, for use in medicine.

4. A functionalized surface prepared according to the method of claim 1 or the functionalized surface according to claim 2 wherein the compound is a pharmaceutically active compound, for use in treatment of bacterial infections, tissue inflammation, or to stimulate tissue regeneration.

5. The method according to claim 1 or the functionalized surface according to claims 2 to 4, wherein
I) the carrier is made of a material comprising silicon, polydimethylsiloxane (PDMS), polyethylene (PE), polyvinyl chloride (PVC), polyurethane (PU), polyethylene terephthalate (PET), poly(methylmethacrylate) (PMMA), polypropylene (PP), ceramics, and/or metal and/or II) the carrier is an implant, preferably an artificial cardiac valve, a catheter, a stent, an intraocular lense, artificial vessels, replacements for tendons & ligaments, tubings, total joint replacements, vascular grafts, a skin replacement, a cardiac pace makers, a finger joint prosthesis, a breast implant, a testicle implant, a gastritic bag, drains, or contact lenses.

6. The method according to claim 1 or 5 or the functionalized surface according to claims 2 to 5, wherein the at least one polymer is a polyanionic polymer, preferably selected from mucin, CM-dextran, carboxymethyl cellulose, alginic acid.

7. The method according to claim 1 or 5 or the functionalized surface according to claims 2 to 5, wherein the at least one polymer is a polycationic polymer, preferably selected from chitosan, Poly-L-Lysine, DEAE-dextran.

8. The method according to claims 1, or 5 to 7 or the functionalized surface according to claims 2 to 7, wherein the cross-linker is selected from an ion, preferably a monovalent, divalent or trivalent ion, more preferably a divalent or trivalent ion, particular preferred a divalent ion.

9. The method according to claims 1, or 5 to 7 or the functionalized surface according to claims 2 to 7, wherein the at least one polymer is an anionic polymer and wherein the cross-linker is a divalent cation, preferably selected from Ba²⁺, Ca²⁺, Mg²⁺, Fe²⁺, Zn²⁺, more preferably from Mg²⁺, Fe²⁺, Zn²⁺.

10. The method according to claims 1, or 5 to 7 or the functionalized surface according to claims 2 to 7, wherein the at least one polymer is a cationic polymer and wherein the cross-linker is a divalent anion, preferably selected from SO₄²⁻, PO₄²⁻, more preferably selected from SO₄²⁻.

11. The functionalized surface for use according to claim 3 or 4, wherein the use comprises exposing the functionalized surface to a medium comprising a physiologically acceptable sodium chloride concentration, preferably a concentration of 90 to 500 mM, more preferably concentration of 100 to 200 mM, most preferably a concentration of 130 to 160 mM.

12. The method according to claims 1, or 5 to 10 or the functionalized surface according to claims 2 to 11, wherein the compound
a) is an anion or cation; and/or
b) is selected from the group consisting of Vancomycin, Tetracycline, Chloramphenicol, Doxorubicin, CM Dextran, Dextran, DEAE Dextran, Atto 488 amine, Atto 488 carboxy (CAS Number: 923585-42-6), and Atto 532 amine or a solvate, hydrate, salt, complex, racemic mixture, diastereomer, enantiomer, tautomer, and isotopically enriched forms thereof and/or
c) is an organic compound and/or
d) is a water soluble drug which preferably has a solubility in water of at least 0.2 μg/ml, more preferably at least 10 μg/ml, particular preferred at least 0.1 mg/ml and/or
e) has a molecular weight of 50 to 12000 Da, preferably 200 to 10000 Da, more preferably 300 to 6000 Da.

13. The method according to claim 1, 5 to 10 or 12, wherein step e) comprises replacing the organic solvent by ultrapure water, wherein the ultra-pure water preferably has an ion content of lower than 1 μg/L more preferred lower than 0.5 μg/L, most preferred lower than 0.05 μg/L.

14. The method according to claim 1, 5 to 10, 12 or 13, wherein the organic solvent is selected from
I) a protic solvent or aprotic solvent; or
II) an alcohol, more preferably an alcohol comprising 1 to 3 hydroxyl groups, most preferably an alcohol comprising 3 hydroxyl groups; or
III) an alcohol comprising 1 to 6 carbon atoms; or
IV) glycerol, methanol, ethanol, propanol, butanol, pentanol, more preferably from glycerol and ethanol, most preferably from glycerol; or
V) hydrocarbon comprising 1 to 8 carbon atoms; or
VI) pentane, hexane, heptane, octane, benzene, toluene, dichloromethane, ethyl acetate, tetrahydrofuran, diethyl ether, preferably hexane.

15. A method for releasing a compound ex vivo comprising exposing the functionalized surface prepared according to the method of claims 1, 5 to 10 or 12 to 14 or the functional surface of claim 2, 5 to 10 or 12 not applied in vivo to a medium comprising a physiologically acceptable sodium chloride concentration, preferably a concentration of 90 to 500 mM, more preferably concentration of 100 to 200 mM, most preferably a concentration of 130 to 160 mM.
